(19) United States
(12) Patent Application Publication
(10) Pub. No.: US 2024/0122673 A1
(43) Pub. Date: Apr. 18, 2024

(54) SURGEON SUPPORT SYSTEM

(71) Applicant: CHASE H. LANO, New Braunfels, TX (US)

(72) Inventor: CHASE H. LANO, New Braunfels, TX (US)

(21) Appl. No.: 18/370,710

(22) Filed: Sep. 20, 2023

Related U.S. Application Data
(60) Provisional application No. 63/408,339, filed on Sep. 20, 2022.

(51) Int. Cl. A61B 90/60; A61G 13/06; A61G 13/10

(52) U.S. Cl. CPC A61B 90/60; A61G 13/06; A61G 13/101

(57) ABSTRACT

Surgeon support systems may be composed of a variety of components and used in a variety of manners to provide support for a surgeon's torso and/or arms. In particular implementations, a surgeon support system may include a base, a vertical support system, and a surgeon support mechanism. The base may be configured to allow a surgeon to move laterally, and the vertical support system may be coupled to the base and have a vertical member that may raise and lower relative to the base. The surgeon support mechanism may be coupled to the vertical support system member near the second end and extending laterally therefrom in a direction perpendicular to the vertical member. In some implementations, a horizontal support system may be coupled to the vertical member near a first end and extend laterally across the base to a second end, where it couples with the surgeon support mechanism.

### RELATED APPLICATIONS

This application claims priority to and the benefit of U.S. Patent Application No. 63/408,339, entitled “Surgeon Support System” and filed on Sep. 20, 2022. This prior application is herein incorporated by reference in its entirety.

### BACKGROUND OF THE INVENTION

Surgeons tend to have back and neck problems due to uncomfortable positioning for extended periods of time during surgery. For instance, surgery often requires the surgeon to be hunched over the patient for hours at a time to be able to access the areas of interest. And for surgeries that require the surgeon to be standing, which are the majority, fatigue, particular of the legs and back, can become a factor.

Surgeries that can be done in the seated position can provide more support to the surgeon. However, with multiple hour operations, even surgeons in seated position can become fatigued. Moreover, the static, uncomfortable posture, whether sitting or standing, can lead to recurring lower back pain and neck pain.

Additionally, surgeons sometimes have to perform detailed work on a patient for extended periods. This can lead to fatigue in their forearms, which can affect their fine motor skills.

These problems are not just related to surgeons, however. Others in the operating room (e.g., surgical first assistant, nurse, operating room technician, surgical technologist, and anesthesiologist) can have similar problems during a single surgery or that develop over the years.

### SUMMARY

The herein described exemplary surgeon support systems have many forms. For example, some are attached to the side rail of an operating table, some are freestanding, and some are attached to the floor of an operating room. Moreover, they each have a variety of features (e.g., providing support to the surgeon, minimizing intrusion into the sterile field, and providing linear and/or rotational adjustments). However, many of the structural aspects of the various embodiments may be implemented in other embodiments.

In one general aspect, a surgeon support system may include a base system, a vertical support, and a surgeon support mechanism. The base system may be attachable to a side rail of an operating table and include a clamp member adapted to be slidably received on an operating table side rail, an elongated member coupled to the clamp member at a first end and extending laterally away therefrom in a direction perpendicular to a received side rail to a second end, and a sleeve mechanism coupled to the elongated member distally from the clamp member, the sleeve mechanism including a vertical sleeve. The vertical support may include a vertical member having a first end configured to be received by the vertical sleeve and extend upward from the first end to a second end distal from the sleeve. The surgeon support mechanism may be coupled to the vertical support and extend laterally therefrom in a direction perpendicular to the vertical member.

In some implementations, the vertical support may include a horizontal member having a first end near the second end of the vertical member. The horizontal member may extend laterally toward the clamp member to a second end, where the surgeon support is coupled to the horizontal member near its second end. Additionally, the horizontal member may be hinged at a midpoint to allow the second end of the horizontal member to rotate laterally relative to the first end of the horizontal member.

In additional implementations, the surgeon support mechanism may include an axle and a surgeon support coupled to the axle. The surgeon support may be attached to the axle, and the vertical support may be configured to receive an end of the axle such that the surgeon support can rotate relative to vertical support. The vertical support may include a rotational locking mechanism to fix the axle at different angles of rotation. Additionally, the vertical support may include a horizontal member having a first end near the second end of the vertical member. The horizontal member may extend laterally toward the clamp member to a second end, where the rotational locking mechanism is located near the second end of the horizontal member.

In certain implementations, the sleeve mechanism may include a sleeve configured to receive the elongated member and slide relative thereto. The sliding of the sleeve mechanism relative to the elongated member may move the vertical support relative to the clamp member.

In particular implementations, the base system is configured such that the elongated member can rotate relative to the clamp member about an axis between the elongated member's ends. For example, the first end of the elongated member may include a disc perpendicular to the axis, and the clamp member include a circular shell for receiving the disc.

In some implementations, the clamp member may include a lateral slot therethrough, and the system may further include a latch member. The latch member may have a first end adapted to receive an end of an operating table side rail and a thin, elongated body that extends through the slot in the clamp member. The elongated body may have a plurality of apertures for receiving pins therein to prevent the clamp member from sliding relative to the latch member.

In some implementations, the base system may be configured such that the elongated member can rotate vertically relative to the clamp member about the elongated member's first end. The clamp member may include a pivot point, and the first end of the elongated member may configured to be coupled to the pivot point.

In particular implementations, the surgeon support system includes a second base system and a second vertical support. The second base system may also be attachable to a side rail of an operating table and include second clamp member, a second elongated member, and a second sleeve mechanism. The second clamp member may be adapted to be slidably received on a side rail of an operating table. The second elongated member may be coupled to the second clamp member at a first end and extend laterally away therefrom in a direction perpendicular to a received side rail to a second end. The second sleeve mechanism may include a vertical sleeve coupled to the elongated member distally from the second clamp member. The second vertical support may include a second vertical member having a first end configured to be received by the second vertical sleeve and extend vertically away from the second sleeve to a second end distal from the second sleeve. The surgeon support may also be attached to the second vertical support and extend laterally therefrom in a direction perpendicular to the second vertical support and towards the first vertical support.

In certain implementations, the first vertical support and the second vertical support are rotatably coupled to an axle of the surgeon support mechanism. The axle may include a telescoping rod. The first vertical support may include a universal joint coupled to one end of the telescoping rod, and the second vertical support may include a universal joint coupled to another end of the telescoping rod.

In another general aspect, a surgeon support system may include a base, a vertical support, and a surgeon support mechanism. The base may be configured to allow the surgeon to stand thereon and to allow a surgeon to move laterally. The base may include wheels for moving the base relative to a floor. The vertical support may be coupled to the base and have a vertical member that may raise and lower relative to the base. The horizontal member may be coupled to the vertical member near a first end and extend laterally across the base to a second end. The surgeon support mechanism may be coupled to the horizontal member near the second end and extend laterally therefrom in a direction perpendicular to the vertical member. The surgeon support mechanism may include an axle and a surgeon support coupled to the axle.

In particular implementations, the vertical support includes a sleeve for receiving the horizontal member. The horizontal member may be horizontally adjusted by sliding through the sleeve.

In certain implementations, the surgeon support mechanism is rotatably coupled to the horizontal member. The horizontal member may include a pivot point near the second end, and one end of the surgeon support mechanism may engage with the pivot point.

In some implementations, the surgeon support system may include a second vertical support, a second vertical member, and a second horizontal member. The second vertical support may be coupled to the base, and the second vertical support having a second vertical member that may raise and lower relative to the base. The second horizontal member may be coupled to the second vertical member. The surgeon support may be coupled to the second horizontal member at a second end.

In some implementations, the first horizontal member and the second horizontal member are rotatably coupled to the surgeon support. The axle, for example, may include a telescoping rod. The first horizontal member may include a universal joint coupled to one end of the telescoping rod, and the second horizontal member may include a universal joint coupled to another end of the telescoping rod.

In an additional general aspect, a surgeon support system comprising may include a first and second vertical support, a first and second horizontal member, and a surgeon support mechanism. The first vertical support system may have a first vertical member that may raise and lower, and the first horizontal member may be coupled to the first vertical member. Similarly, the second vertical support system may have a second vertical member that may raise and lower, and the second horizontal member may be coupled to the second vertical member. The surgeon support mechanism may be coupled to the first horizontal member at one end and to the second horizontal member at a second end. The surgeon support may extend laterally in a direction perpendicular to the vertical members and include an axle and a surgeon support coupled to the axle.

In certain implementations, each vertical support system includes a sleeve for receiving a respective horizontal member. The horizontal members may be horizontally adjustable by sliding through the sleeves. The surgeon support may be rotatable relative to the horizontal members.

In some implementations, the axle may include a telescoping rod. Additionally, the first horizontal member may include a universal joint coupled to one end of the telescoping rod, and the second horizontal member may include a universal joint coupled to another end of the telescoping rod.

The above-described implementations have variety of features. For example, the implementing structure may be offset from the surgeon's critical work area. This assists in maintaining sterility and allows for improved surgeon movement and sight, even while standing in one position. In some implementations even, a surgeon may be able to move side to side, which may be required for a procedure and/or comfort, while still maintaining support. Additionally, the various implementations have linear and/or rotational adjustments, which will help to customize the system for each surgeon and/or surgical procedure. Various implementations may have one or more features.

Additional aspects and features of the inventive concepts will be revealed to those of ordinary skill in the art upon review of the following detailed description and the accompanying drawings. The aspects and features of a particular embodiment are not limited to that embodiment unless specified.

### BRIEF DESCRIPTION OF THE DRAWINGS

For a more complete understanding of the present invention and its implementations, and the features thereof, reference is now made to the following descriptions taken in conjunction with the accompanying drawings, in which:

FIG. 1 is a line drawing illustrating an example surgeon support system.

FIGS. 2A-2C are line drawings illustrating an example clamp member and elongated member for a base system of surgeon support system similar to the surgeon support system in FIG. 1.

FIG. 3-3A are line drawings illustrating an example sleeve mechanism for a surgeon support system similar to the surgeon support system in FIG. 1.

FIG. 4 is a line drawing illustrating the interaction between the vertical support and the surgeon support mechanism in FIG. 1 in more detail.

FIGS. 5-5D are line drawings illustrating selected portions of another base system for a surgeon support system similar to the surgeon support system in FIG. 1.

FIGS. 6-6B are line drawings illustrating another example base system for a surgeon support system similar to the surgeon support system in FIG. 1.

FIGS. 7-7B are line drawings illustrating selected components of another example base system 700 for a surgeon support system similar to the surgeon support system in FIG. 1.

FIG. 8 is a line drawing illustrating selected components of another example base system.

FIGS. 9-9C are line drawings illustrating another example surgeon support system.

FIGS. 10-10B are line drawings illustrating an example vertical support for a surgeon support system similar to the surgeon support system in FIG. 1.

FIGS. 11A-11B are line drawings illustrating an example surgeon support mechanism for use with a surgeon support system similar to the surgeon support system in FIG. 1.

FIGS. 12-12c are line drawings illustrating an example interaction between a vertical support and a surgeon support mechanism.

FIG. 13 is a line drawing illustrating another example surgeon support system.

FIG. 14 is a line drawing illustrating an additional example surgeon support system.

FIG. 15 is a line drawing illustrating an further example surgeon support system.

FIG. 16 is a line drawing illustrating an another example surgeon support system.

FIG. 17 is a line drawing illustrating selected components of an example surgeon support system.

FIG. 18 is a line drawing illustrating another example surgeon support system.

FIG. 19 is a line drawing illustrating an example frame for surgeon support mechanism.

FIGS. 20-23 are line drawings illustrating example pads for a surgeon support mechanism.

FIGS. 24A-24C are line drawings illustrating another example surgeon support system in use.

### DETAILED DESCRIPTION

While the inventive concepts are much more basic than any particular implementation, one skilled in the art can gather a partial appreciation for some of the possible benefits of the broader concepts and possible interplay between various elements of the concepts in the course of considering example implementations, some of which are described in detail below.

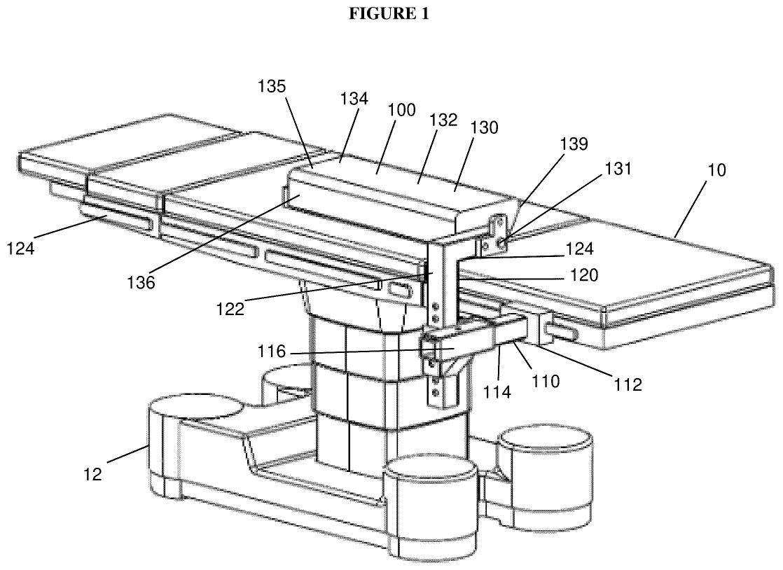

FIG. 1 illustrates an example surgeon support system 100. Among other things, surgeon support system 100 includes a base system 110, a vertical support 120, and a surgeon support mechanism 130.

As illustrated, system 100 is coupled, via base system 110, to an operating table 10, which has a base 12 and a side rail 14. As explained below, surgeon support system 100 may be coupled to and decoupled from side rail 14, which may provide one adjustment for the surgeon support system 100.

Base system 110 includes a clamp member 112, an elongated member 114, and a sleeve mechanism 116. Clamp member 112 is configured to be slidably received on rail 14 of operating table 10 and secured thereon. Elongated member 114 is coupled to clamp member 112 and extends laterally away from side rail 114. Sleeve mechanism 116 is configured to be slidably received on elongated member 112 and coupled thereto.

Vertical support 120 includes a vertical member 122 and a horizontal member 124. Vertical member is configured to be slidably received in sleeve mechanism 116 and secured thereto. Horizontal member 124 is coupled to vertical member and extends laterally therefrom toward side rail 14.

Surgeon support mechanism includes an axle 131 (partially viewable here) and a surgeon support 132. As explained below, axle 131 may be coupled to vertical support 120 such that the axle does not rotate relative to the vertical support but allows surgeon support 132 to rotate relative to the axle. In other embodiments, axle 131 may be coupled to vertical support 120 such that the axle does rotate relative to the vertical support but surgeon support 132 rotates with the axle.

Surgeon support 132 includes a base (not viewable here) and a padded structure 134 over the base. Surgeon support 132 rotates relative to vertical support 120 to provide different surface areas for a surgeon to rest part of his/her body on. For example, in the illustrated example, surgeon support 132 has a large area 135, a first small area 136, and a second small area 139, which is rounded at the transition from the large area and tapers inwardly. As illustrated, a surgeon could rest his/her for forearms on large area 135 (e.g., if doing detailed work) or a body part on their frontal axis (e.g., chest, stomach, or hips) on small area 136. But if the pad was rotated 90 degrees counterclockwise, for example, a surgeon could rest his torso on large area 135 and his/her armpits on small area 139. Additionally, if the pad was lowered in this orientation, the surgeon could rest his torso on the large area and his elbow or the upper part of his forearms on the small area. Moreover, in some cases, a surgeon may just rest his torso on the pad or a part of his arms on the pad. Surgeon support system 130 may oriented at various angles in particular embodiments.

Surgeon support 132 may be about 24 inches wide (measured along axle 131), which is wider than the shoulders for most males. However, surgeon support 132 may be narrower than this for particular surgeons. Additionally, in some embodiments, surgeon support may be much wider (e.g., 36 inches or 48 inches). This will allow the surgeon to move along the side rail of the operating table (e.g., to obtain different view and/or to perform different procedures during an operation).

Surgeon support system 110 may be made over various materials. For example, base system 110 and vertical support 120 may be made of stainless steel, aluminum, plastic or composite. Padded structure 134 could, for example, include a layer of foam covered by a flexible plastic (e.g., vinyl) cover.

System 100 has a variety of features. For example, system 100 allows a surgeon to receive support for their torso (e.g., chest or pelvis), which may reduce back pain, and/or arms (e.g., upper arms, forearms, or wrists), which may promote hand stability, while placing minimal (or possibly) no structure over the sterile field. This will assist in keeping the operating filed sterile. Moreover, most of the hardware is to the side and/or behind the surgeon, and nothing prevents his/her horizontal movement along the surgeon support 132. Thus, there is minimal effect on the surgeon's sight lines, arm positions, and body positions. There is not, for instance, a singular position at which the surgeon is supposed to contact the surgeon support mechanism to receive support. Moreover, a surgeon may move and/or rearrange their feet as needed. As another example, system 100 may be attached to operating table 10 at various positions relative to the operating table. Thus, system 100 may be positioned at advantageous points for a particular surgery. As an additional example, system 100 allows surgeon support 132 to be raised and lowered, which may be useful for surgeons of different sizes and/or different types of surgeries (e.g., shoulder versus abdominal). As a further example, system 100 allows surgeon support 132 to be brought closer to and away from operating table 10, which may be useful for different type of surgeries (e.g., arm or abdominal). As another, system 100 allows surgeon support to 132 to be reoriented to present various support aspects to the surgeon. In this embodiment, surgeon support 132 can be adjusted in four dimensions (i.e., along the rail, in height, in closeness to the rail, and in rotation).

Additionally, each adjustment for surgeon support 132 is isolated in this embodiment. That is, each adjustment only moves surgeon support 132 in one direction. For example, adjusting the surgeon support in distance from the side rail of the operating table may performed without adjusting the surgeon support's height. This allows the surgeon support to achieve an ideal position for a surgeon once all the preferred adjustments are made. Different adjustments, however, are provided in different embodiments, as described below.

Other embodiments could have fewer and/or additional components. For example, an embodiment could have a second base system with an attached vertical support that is coupled to the other end the surgeon support system. Additionally, a vertical support may not have a horizontal member in some embodiments. Thus, it may be enough for the surgeon support mechanism to be moved away from the side rail and up. Various angular adjustment features are also possible, several of which will be discussed in detail below.

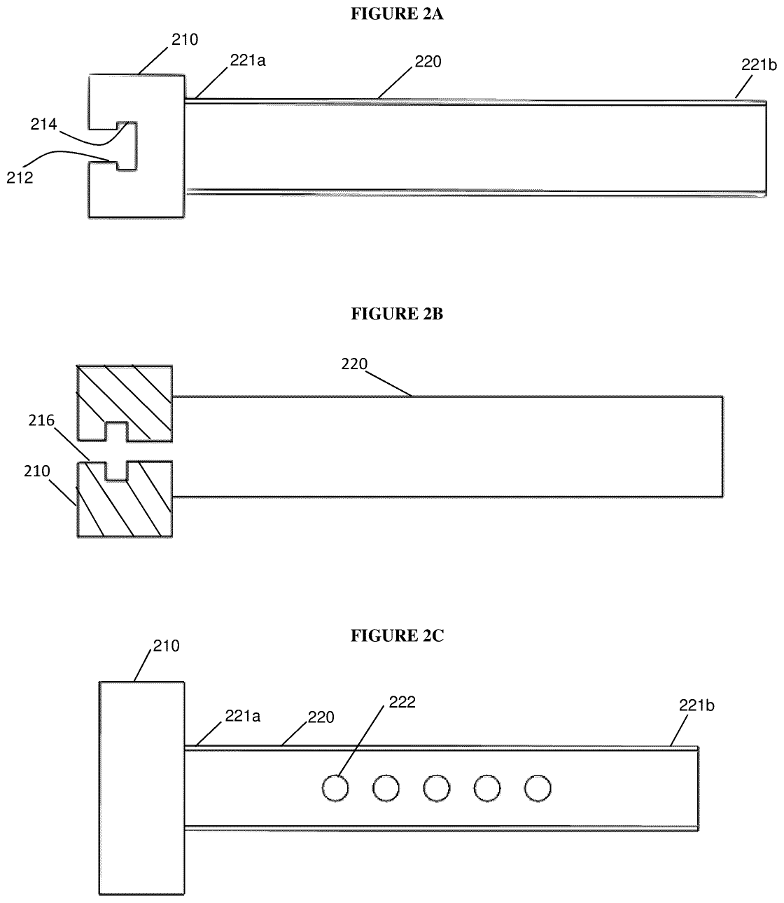

FIGS. 2A-2C illustrate an example clamp member 210 and elongated member 220 for a base system for a surgeon support system similar to surgeon support system 100. FIG. 2A shows a side view of clamp member 210 and elongated member 220, FIG. 2B shows a partially cross-sectioned side view of the members, and FIG. 2C shows a top view of the members.

As illustrated in FIG. 2A, clamp member 210 includes an outer notch 212 and an inner notch 214, which is wider than outer notch 212. Inner notch 214 allows for the receiving of a side rail of an operating table, and outer notch 212 allows for capturing of the side rail (e.g., so that clamp member 210 cannot be pulled laterally away from the side rail).

FIG. 2B shows a side view of clamp member 210 and elongated member 220 with clamp member 210 sectioned through a channel 216. (Clamp member 210 may have a similar channel on the other side of elongated member 220.) Clamp member 210 may be further secured to the side rail by posts (not shown) that extend through channel 216 and frictionally engage the rail. The posts may be smooth, threaded, or otherwise.

Elongated member 220 is coupled to clamp member 220 at a first end 221a and extends laterally therefrom to a second send 221b when clamp member is coupled to the side rail of an operating table. Note that laterally, and for that matter horizontally, does not mean perfectly horizontal. Some operating tables allow for tilt along their long axis. Thus, elongated member 220 may be angled slightly above or slightly below perfectly horizontal, typically up to 15 degrees, but up to 30 degrees in some instances. Elongated member 220 may be coupled to clamp member 210 by being integrally formed therewith, by being welded thereto, by being bolted thereto, or by other appropriate technique.

Elongated member 220 includes a number of apertures 222 therein. Apertures 222 allow elongated member 220 to receive one or more locking members (e.g., a spring loaded pin or a threaded bolt) pins to lock a sleeve mechanism (e.g., sleeve mechanism 116) relative thereto.

As illustrated, elongated member 220 has a square cross-section. But elongated member 220 may have other cross-sections (e.g., circular, oval, rectangular, etc.) in other embodiments.

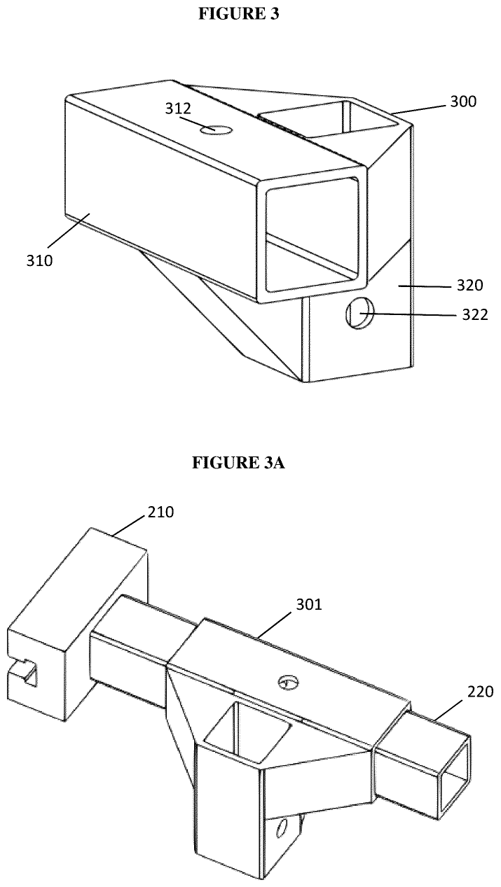

FIGS. 3-3A illustrates an example sleeve mechanism 300 for a surgeon support system similar to surgeon support system 100. Among other things, sleeve mechanism 300 includes a first sleeve 310 and a second sleeve 320.

Sleeve 310 is configured to slidably receive elongated member 220, which is coupled to clamp member 210. Sleeve 310 may thus be slid relative to clamp member 210, moving the sleeve mechanism closer to and further away from the clamp member. Sleeve 310 includes an aperture 312. Aperture 312 is configured to receive a locking member (e.g., a spring loaded pin or a threaded bolt) that is also received in an aperture of elongated member 220 to secure the sleeve mechanism relative to the elongated member.

Sleeve 320 is configured to slidably receive a vertical member (not shown). The vertical member may thus be slid relative to sleeve mechanism 360, moving the top of the vertical member closer to and further away from sleeve 320. Sleeve 320 includes an aperture 322. Aperture 322 is configured to receive a pin (e.g., a spring-loaded pin or a bolt) that is also received in the vertical member to secure the vertical member relative to the sleeve mechanism.

As illustrated sleeve 310 and sleeve 320 have square cross-sections. Sleeve 310 and 320 may have other cross sections in other embodiments (e.g., rectangular, circular, oval, etc.). Additionally, sleeve 310 and sleeve 320 are coupled to each other at a roughly 90 degree angle. Sleeve 310 and sleeve 320 may, however, be coupled to each other at other angles in other embodiments. Sleeve 310 and sleeve 320 may be coupled to each other by being integrally formed with each other or by being formed separately and then being attached (e.g., by welding, brazing, or adhesion).

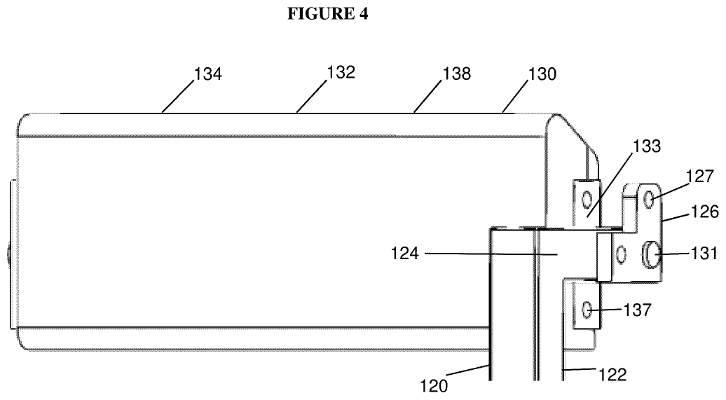

FIG. 4 illustrates the interaction between vertical support 120 and surgeon support mechanism 130 of surgeon support system 100 in more detail. As can be seen, coupled to the end of horizontal member 124 is a locking mechanism 126. Locking mechanism 126 is adapted to receive axle 131 of surgeon support mechanism 130. Additionally, locking mechanism 126 includes apertures 127.

Surgeon support 132 includes a frame 133 upon which padded structure 134 is mounted. Frame 133 includes an aperture therethrough (not viewable here) for receiving axle 131. Additionally, frame 133 includes apertures 137 therein. Apertures 137 correspond to apertures 127 in locking mechanism 126 as surgeon support 132 is rotated around axle 131. By inserting a locking mechanism through one of apertures 127 and into one of apertures 137, surgeon support 132 may be locked in a rotational position. Here, three locking positions are possible that allow the use of padded structure 134—the one shown, one 90 degrees clockwise from that, and one 90 degrees clockwise from that. Fewer or additional rotational locking positions may be used in other embodiments.

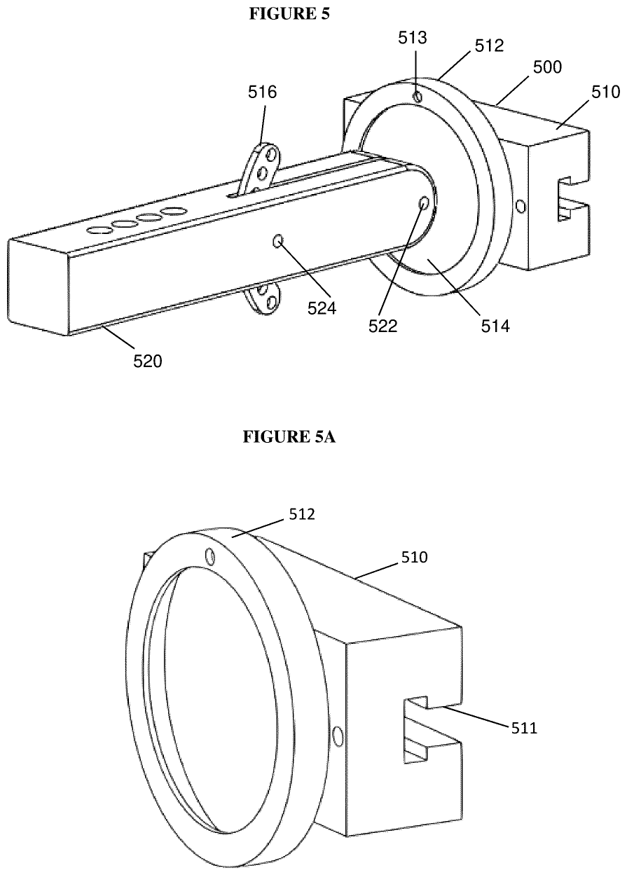

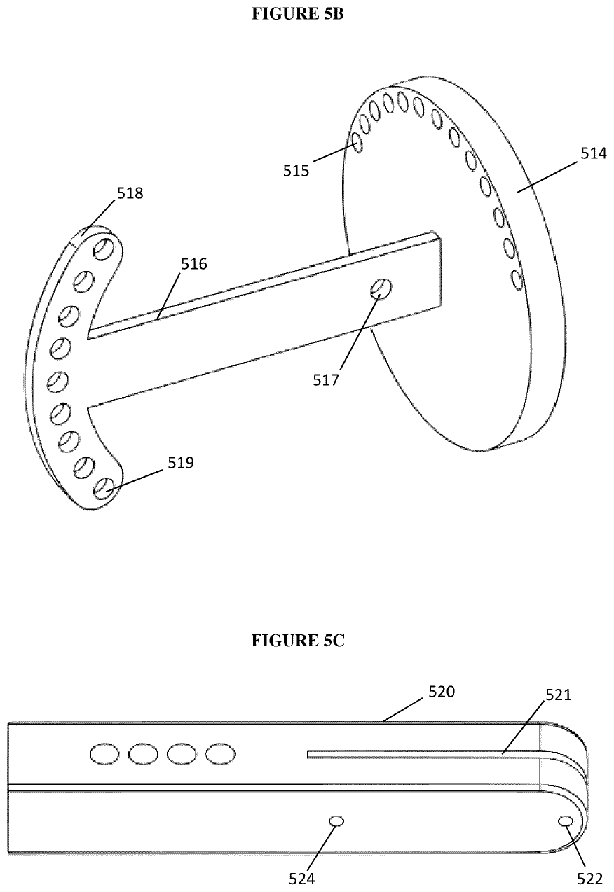

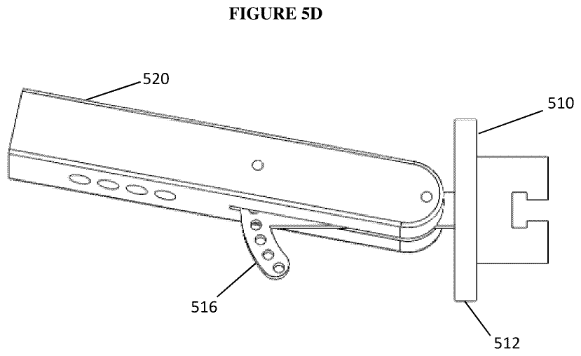

FIGS. 5-5D illustrate selected portions of another example base system 500 for a surgeon support system similar to surgeon support system 100. Among other things, base system 500 includes a clamp member 510 and an elongated member 520.

Clamp member 510 includes a notch 511 for receiving a side rail of an operating table. Additionally, clamp member 510 includes a circular race 512. As illustrated, race 512 captures a disc 514 and allows it to rotate therein. Race 512 includes an aperture 515 for receiving a locking member to secure disc end 514 relative to the race. Disc 514 includes a number of apertures 515 to lock disc 514 at various rotatable positions relative to the race due to a locking member being inserted in aperture 513.

Coupled to the circular disc is an arm 516, which extends laterally away therefrom. Arm 516 includes an aperture 517, which acts as a pivot for elongated member 520, and an arced member 518 at its distal end. Arced member 518 includes a number of apertures 519 along its arc.

Elongated member 520 includes a slot 521 that is sized to allow arm 516 to pass through. Additionally, elongated member 520 includes an aperture 522 that may be pinned to aperture 517 to allow elongated member 520 to pivot relative to arm 516. Elongated member 520 generally rotates vertically, but due to the ability to change the rotational position of disc 514, elongated member may rotate off vertical, by up to 30 degrees, for example. FIG. 5D shows elongate member 520 and arm 516 rotated relative to clamp member 510 (i.e., by arm 516 being rotated in race 512) and elongated member 520 being rotated relative to arm 516.

Elongated member 520 also includes an aperture 524. Aperture 524 allows elongated member 520 to be fixed in a position of rotation relative to arm 516 by receiving a locking member (e.g., a spring loaded pin or a threaded bolt) through aperture 524 and one of apertures 519 in arced member 518.

Base system 500 allows for a rotary adjustment for an attached surgeon support, such as, for example, surgeon support 130. That is, the long axis of the surgeon support may be rotated in the vertical plane running therethrough. Some operating tables allow for tilted positions of the patient when viewing them from head to toe, resulting in the patient's feet being above the head (commonly known as the Trendelenburg position) or the patient's beet being below the head (commonly known as the reverse Trendelenburg position). Rotation of an operating table to a Trendelenburg position would cause a surgeon support to assume a slanted position, which may not be optimal for a surgeon. But rotating elongated member 520 via disc 514 in race 512 allows for amelioration of some, or even all, of this slant.

Additionally, some operating tables allow for a tilted position of the patient when viewing them along their height (e.g., one shoulder would be raised above another). Rotation of an operating table in this manner would cause a surgeon support to assume a slanted position, which may not be optimal for a surgeon. But rotating elongated member 520 relative to arm 516 allows for amelioration of some, or even all, of this slant.

Note that both the rotary adjustments available in base system 500 may be achieved independently of each other. Thus, they do not interfere with each other. If an operating table has simultaneous Trendelenburg and lateral rotations, the isolated adjustments should make it possible to achieve a horizontal surgeon support member (if that is what the surgeon desires).

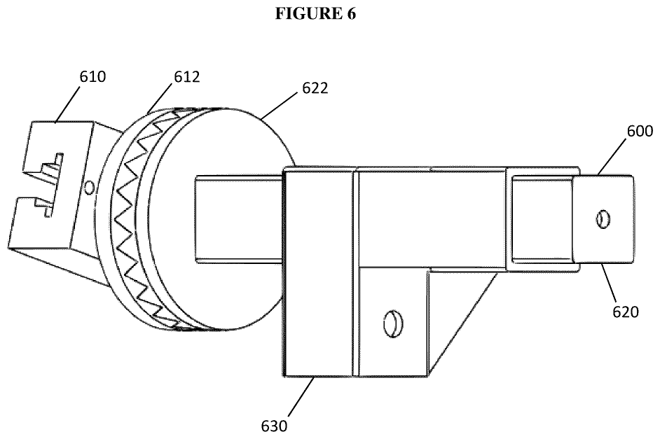

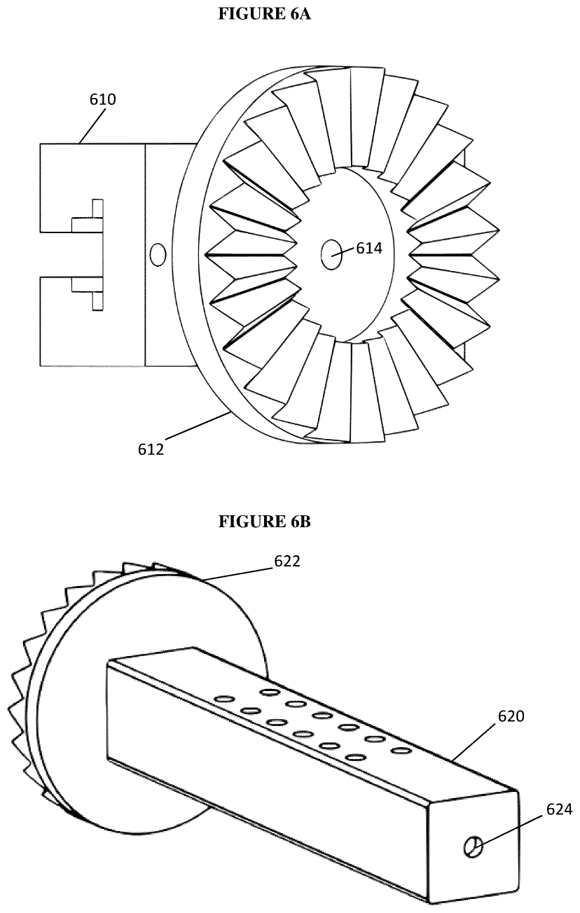

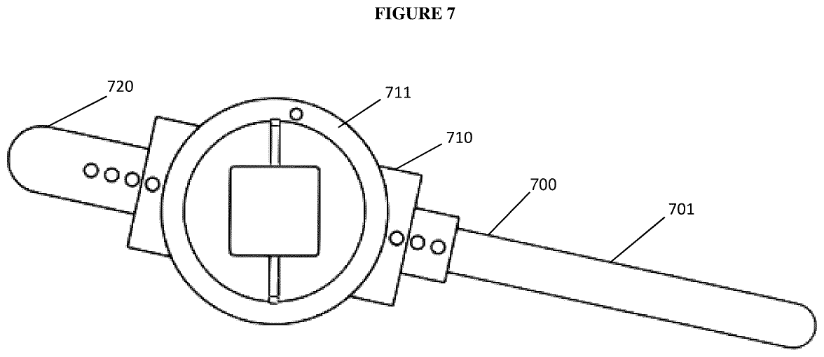

FIGS. 6-6B illustrate another example base system 600 for a surgeon support system, such as, for example, surgeon support system 100. Base system 600 includes a clamp member 610, an elongated member 620, and a sleeve mechanism 630. Among other things, base system 600 allows elongated member 620 to be adjusted in rotation relative to clamp member 610, to accommodate Trendelenburg positions.

As illustrated, clamp member 610 and elongated member 620 each include half of a Hirth joint—Hirth component 612 and Hirth component 622, respectively. Clamp member 610 also includes an aperture 614 for receiving a threaded member (not shown), and elongated member includes an aperture 624 for receiving the same threaded member. Aperture 624 may or may not be threaded.

To adjust the rotation of elongated member 620 relative to clamp member 610, elongated member 620 is separated from clamp member 610 and rotated to the appropriate angle. Then, Hirth component 622 is meshed with Hirth component 612. Note the Hirth component 612 allows rotational positioning at 15 degree increments. Thus, while the exact rotational position may not be achievable, it should be close enough to allow for surgeon preference. Other rotational increments may be used in other embodiments. After meshing the Hirth components together, a threaded member (typically with a hand grip on one end) may be inserted through aperture 624 and aperture 614 and secured to clamp member 610, axially engaging elongated member 620 to clamp member 610. The threaded member should not interfere with the sliding of sleeve mechanism 630 relative to elongated member 610.

Aperture 614 may end inside clamp member 610 or it may extend all the way through the clamp member. If aperture 614 extends all the way through the clamp member, then the threaded member can be tightened to the point where it makes contact with the side rail, which may help keep the base system secured to the side rail.

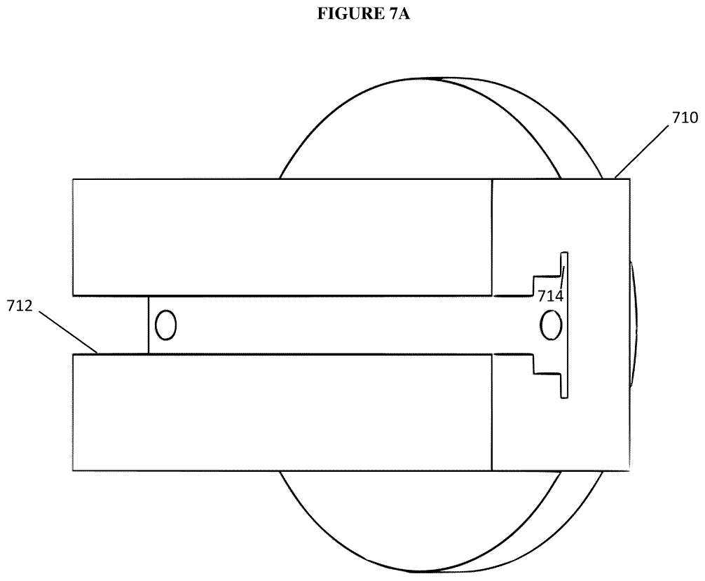

FIGS. 7-7B illustrate selected components of another example base system 700. Base system 700 includes a clamp member 710 and a latch 720. Base system 700 is useful for securing clamp member 710 to at a position on a side rail 701 when the base system is used in a Trendelenburg position. As illustrated, base system 700 is shown using a race 711 to allow rotary adjustment relative to a slanted rail, but base system 700 could use other rotary adjustment mechanisms (e.g., Hirth joint).

Clamp member 710 includes a notch 712 for receiving the side rail of an operating table and an inner notch 714 for receiving latch 720. Latch 720 includes an elongated portion 722 with a receiving member 724 at one end. Elongated portion 722 is configured to fit within inner slot 714, and receiving member 724 is configured to receive an end of side rail 701.

Elongated member 722 includes a number of apertures 726 through which the posts used to secure clamp member to the side rail may also extend. By this, clamp member 710 will be unable to slide relative the side rail even if the friction between the posts and the rail is insufficient to prevent sliding.

Note the latch 720 may be used on either end of side rail 701. Thus, clamp member 710 may be prevented from sliding for either Trendelenburg position.

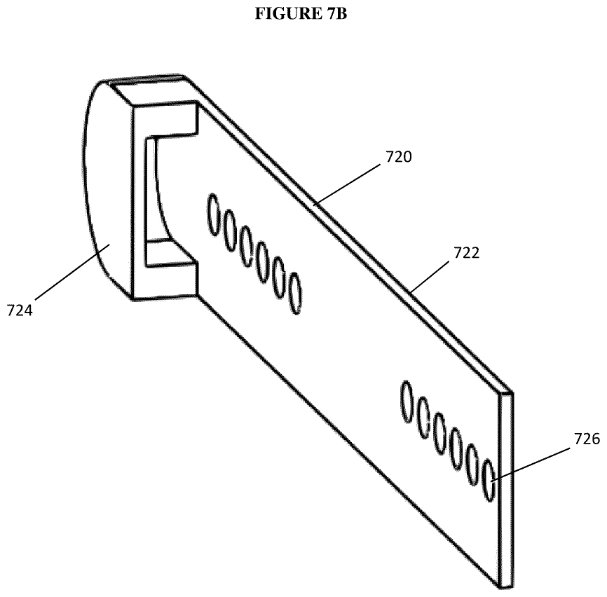

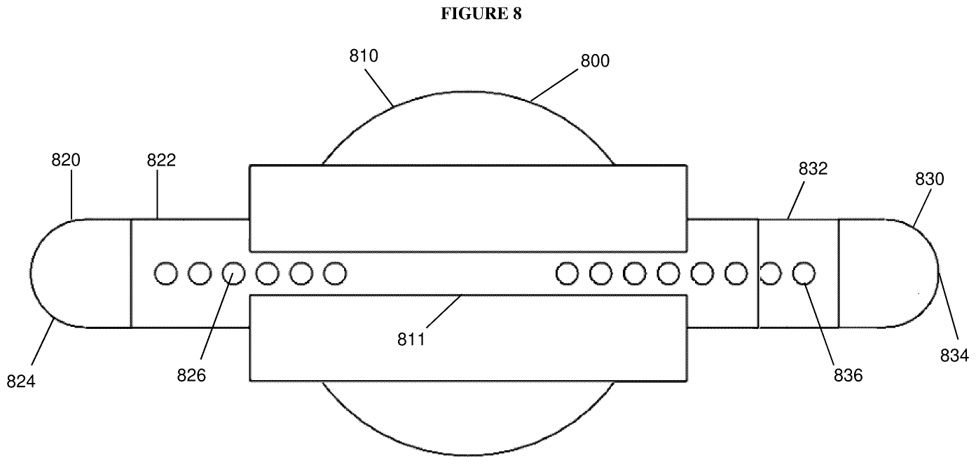

FIG. 8 illustrates selected components of another example base system 800. Base system 800 include a clamp member 810, a latch 820, and a latch 830. Similar to base system 700, base system 800 is useful for securing clamp member 810 to at a position on a side rail when the base system is used in a Trendelenburg position. However, base system 800 secures for either Trendelenburg position. This is especially useful if an operation requires switching between Trendelenburg positions midstream.

Base member 810 includes a notch 811 for receiving the side rail of an operating table and receiving latches 820, 830. Latch 820 includes an elongated portion 822 with a receiving member 824 at one end. Latch 830 includes an elongated portion 832 with a receiving member 834 at one end. Elongated portions 820, 830 are configured to simultaneously fit beside each other within inner notch 811, and receiving members 824, 834 are configured to each receive an end of a side rail. In particular embodiments, one latch may have a hollow area deeper than the other so that when they overlap each other, both latches can make contact with the back of the rail ends.

Elongated member 822 and elongated member 832 each include a number of apertures 826, 836 through which the posts used to secure clamp member 810 to the side rail may also extend. By this, clamp member 810 will be unable to slide relative the side rail even if the friction between the posts and the rail is insufficient to prevent sliding. Moreover, this will prevent sliding of the clamp member no matter which end of the operating table is elevated.

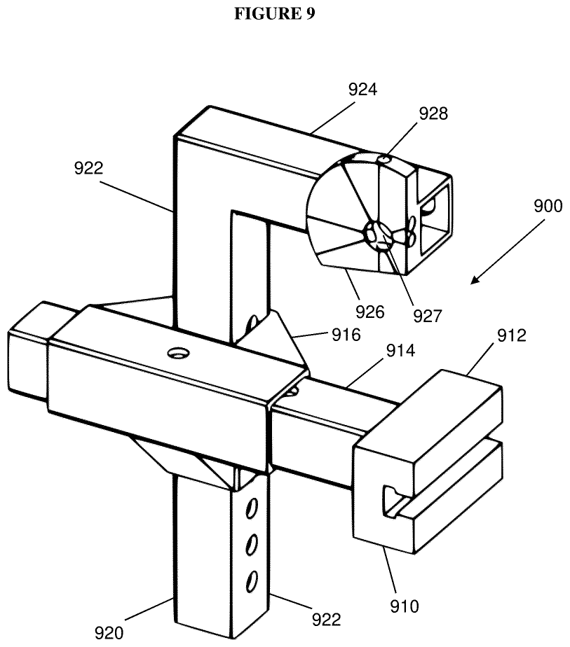

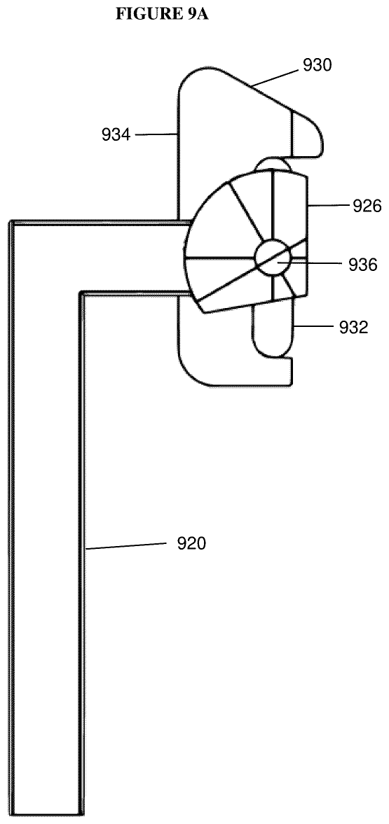

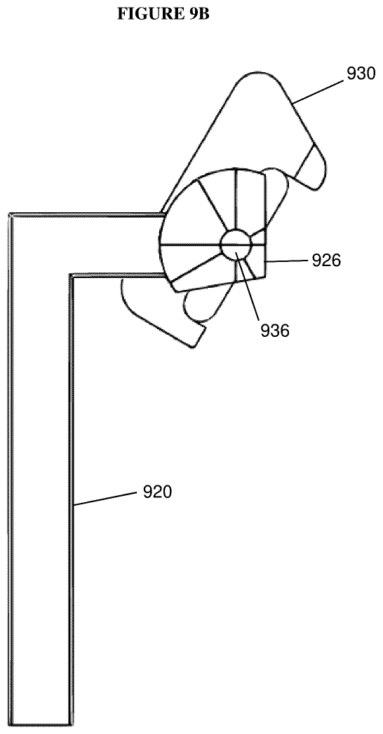

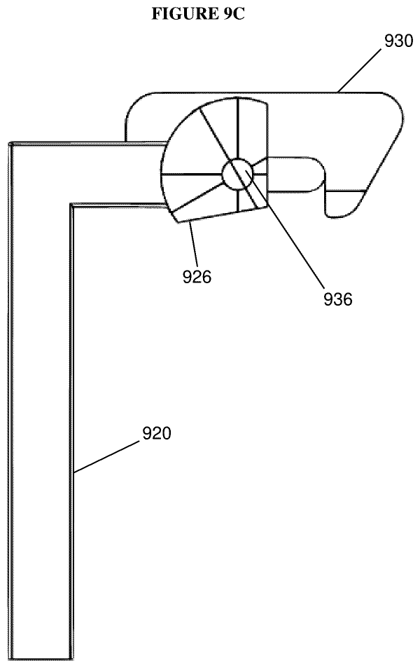

FIGS. 9-9C illustrate another example surgeon support system 900. Surgeon support system 900 includes a base system 910, a vertical support 920, and a surgeon support mechanism 930.

Base system 910 includes a clamp member 912 that is configured to be slidably received on a side rail of an operating table. Extending laterally from clamp member 912 is an elongated member 914. Slidably received on elongated member is a sleeve mechanism 916.

Vertical support 920 includes a vertical member 922 that is received at a lower end in sleeve mechanism 916, which allows the vertical member to slide relative thereto. Coupled to the upper end of vertical support 922 is a horizontal member 924, which projects back toward clamp member 910. At the end of horizontal member 924 is a locking mechanism 926, which locks surgeon support mechanism 930 at different rotational orientations. Locking mechanism 926 includes an aperture 927 therethrough.

Surgeon support mechanism 930 includes a frame 932 and a pad 934 mounted on the frame. Projecting out of frame 932 is an axle 936, which is received in aperture 927 to allow rotation of surgeon support 930 relative to vertical support 920.

Locking mechanism 926 also includes a number of apertures 928 along its periphery. Apertures 928 are adapted to receive a pin therethrough, which may also inserted into a radial aperture in axle 936 (not viewable here). By inserting a pin through one of apertures 928 and into the radial aperture in axle 936, the axle may be locked in rotational motion.

FIGS. 9A-9C specifically show the rotational motion of surgeon support 930 about axle 936. Surgeon support 930 may be locked in rotation at any of these points, as well as others.

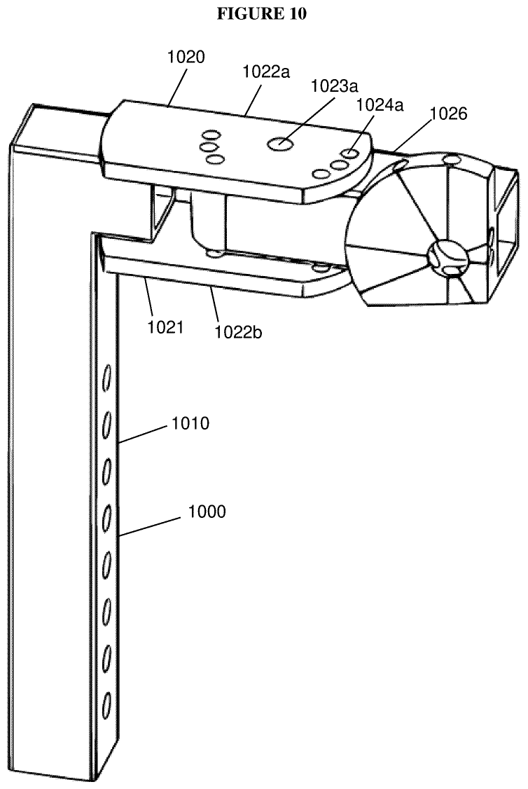

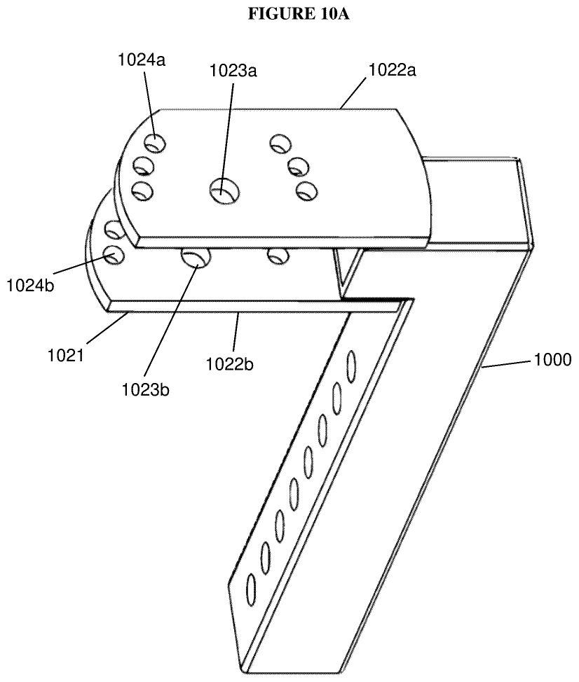

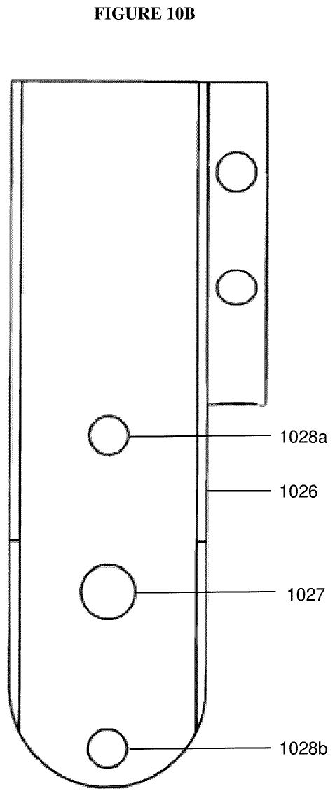

FIGS. 10-10B illustrate an example vertical support 1000 for a surgeon support system, such as, for example, the surgeon support system 100. Among other things, vertical support 1000 includes a vertical member 1010 and a horizontal member 1020.

Horizontal member 1020 includes two components—a base 1021 and a swing arm 1026. Base 1021 includes an upper plate 1022a and a lower plate 1022b, which form a slot for receiving a portion of swing arm 1026. Upper plate 1022a and lower plate 1022b respectively include an aperture 1023a and an aperture 1023b. Swing arm 1026 includes an aperture 1027 that is aligned with aperture 1023a, 1023b and pinned to allow swing arm 1026 to rotate relative to base 1021. Thus, an attached surgeon support mechanism may be adjusted about the longitudinal axis.

Plates 1022a and 1022b also respectively include apertures 1024a and 1024b, and swing arm 1026 includes apertures 1028a, 1028b. Apertures 1028a, 1028b align with various ones of apertures 1024a, 1024b as swing arm 1026 is rotated relative to base 1021. By placing a pin through one of apertures 1028a, 1028b and into one of one of apertures 1024a, 1024b, swing arm 1026 may be locked in position relative to base 1021.

Vertical support 1000 allows a surgeon support mechanism attached to the vertical support to be adjusted rotationally in a horizontal plane, assuming vertical member is vertical. During surgery, a surgeon's hips are not always parallel to the side rails of an operating table. In these instances, the surgeon may need to lean on a pad that is parallel with the desired placement of his/her hips. Vertical support 1000 allows this to occur.

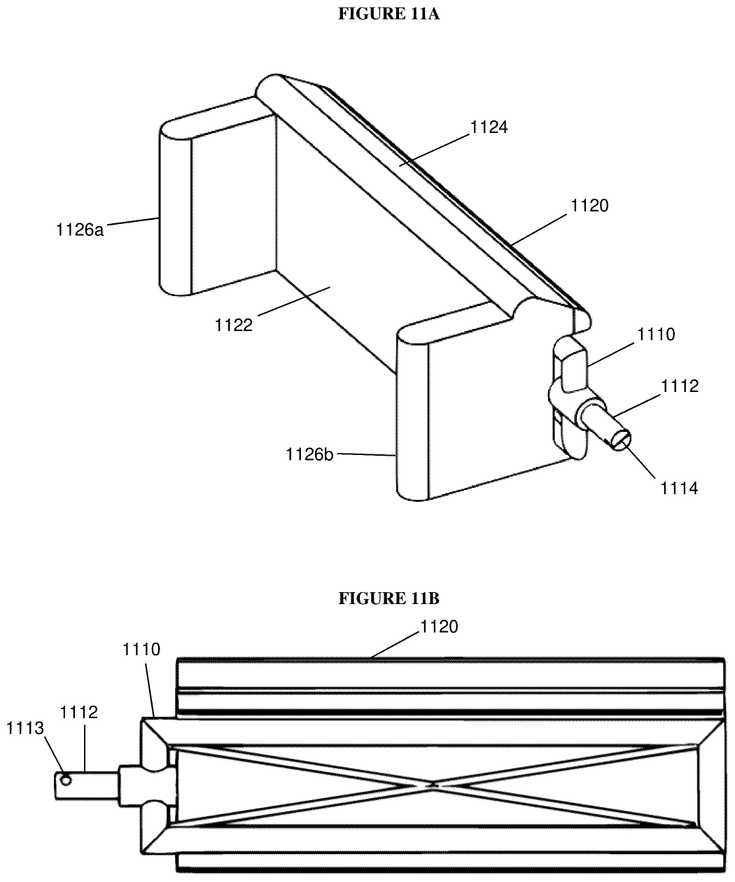

FIGS. 11A-11B illustrate an example surgeon support mechanism 1100 for use with a surgeon support system similar to the surgeon support system 100. Among other things, surgeon support system 1100 includes a frame 1110 and a surgeon support 1120.

As illustrated, frame 1110 is generally rectangular in outer shape. But frame 1110 may have other outer shapes (e.g., square, circular, oval, etc.) in other implementations. Additionally, frame 1110 may allow surgeon supports of various configuration to be coupled thereto. However, some embodiments may use an integrated frame/surgeon support, possibly even with the frame being internal to the surgeon support.

At one end, frame 1110 includes an axle 1112, which has a radial aperture 1113 therein. Axle 1112 may be received in an aperture of a vertical support to allow surgeon support mechanism to rotate relative thereto. Aperture 1113 allows a pin to inserted therein (e.g., slidably or threadably) to secure surgeon support mechanism in rotation. A demarcation 1114 (e.g., a mark, a groove, a protuberance, etc.) on the end of axle 112 shows the alignment of aperture 113.

Surgeon support 1120 includes a large flat area 1122, on which a surgeon may rest his/her torso or forearm, depending on rotation of surgeon support mechanism 1100. Additionally, surgeon support mechanism includes a shallow, tapered top 1124 and side members 1126. Top 114 may be useful for a surgeon to rest his/her forearms on and/or to rest his/her armpits on. Side members 1126 may be useful for a surgeon to rest one side of his torso (e.g., hips).

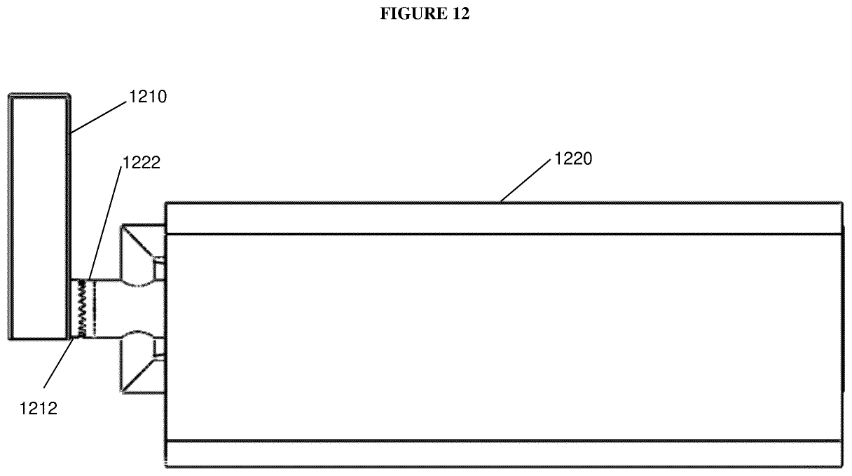

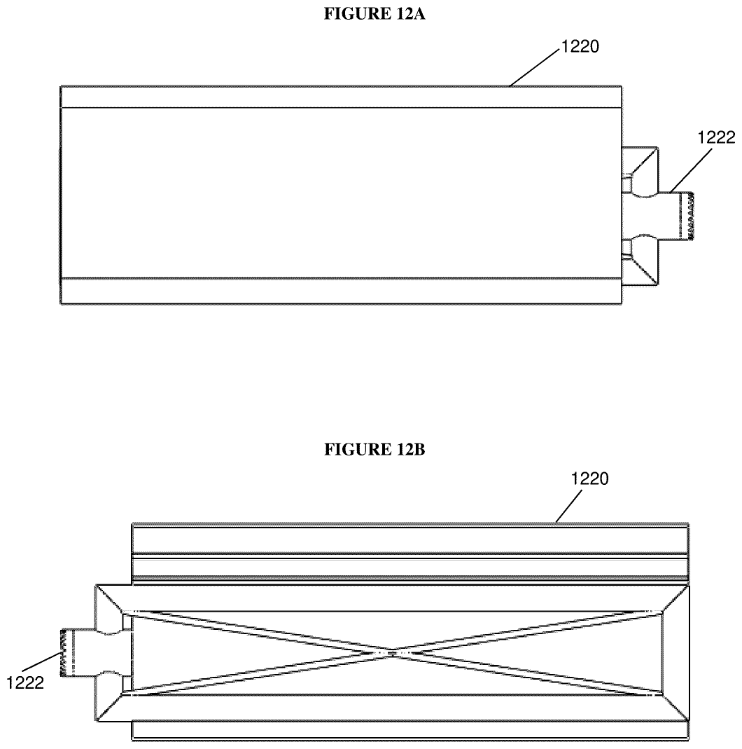

FIGS. 12-12C illustrate an example interaction between a vertical support 1210 and a surgeon support mechanism 1220. FIG. 12 shows surgeon support mechanism 1220 in one orientation relative to vertical support 1210, and FIG. 12C shows surgeon support mechanism 1220 in another orientation relative to vertical support 1210.

As illustrated, each of vertical support 1210 and surgeon support mechanism 1220 includes half of a Hirth joint—Hirth component 1212 and Hirth component 1222, respectively. Vertical support 1210 also includes an aperture 1214 for receiving a threaded member (not shown) and allowing it to pass into Hirth component 1222. The aperture in Hirth component 1212 may or may not be threaded.

To adjust the rotation of elongated member surgeon support mechanism 1220 relative to vertical support 1210, surgeon support mechanism 1220 is separated from vertical support 1210 and rotated to the appropriate angle. Then, Hirth component 1222 is meshed with Hirth component 1212. Note that Hirth components 1212 allows rotational positioning at discrete increments. Thus, while the exact position may not be achievable, it should be close enough to allow for surgeon comfort. After meshing the Hirth components together, a threaded member (typically with a hand grip on one end) may be inserted through aperture 1214, through Hirth component 1212, and into the aperture in Hirth component 1222 and secured thereto by axial rotation, axially engaging surgeon support member 1220 to vertical member 1210.

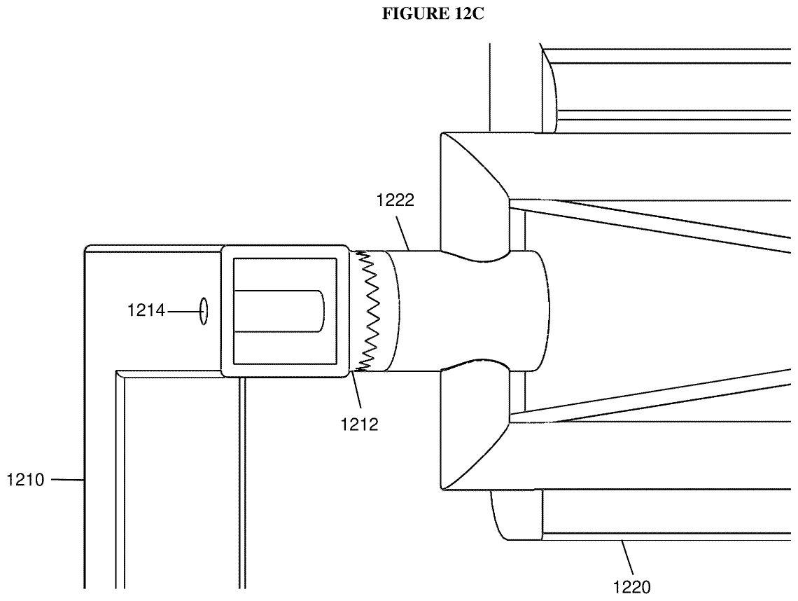

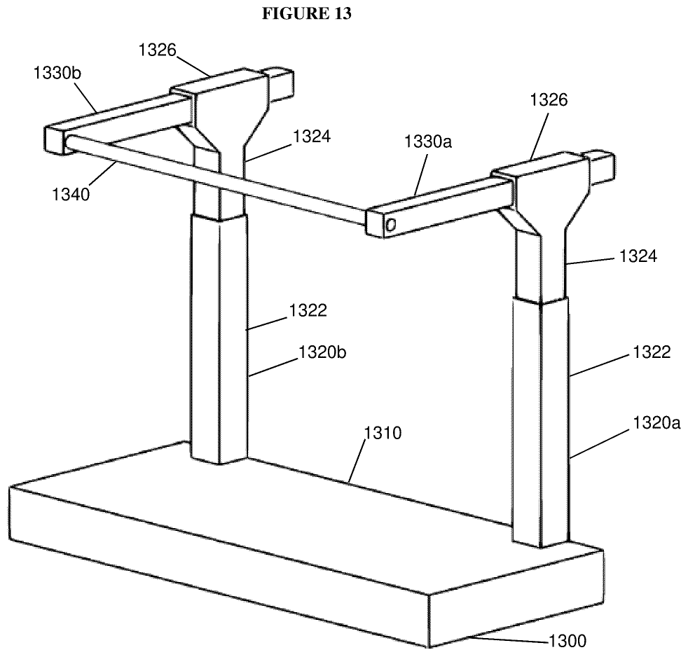

FIG. 13 illustrates another example surgeon support system 1300. Among other things, surgeon support system 1300 includes a base 1310, vertical supports 1320, horizontal supports 1330, and surgeon support mechanism 1340.

Base 1310 is configured to support the weight of a surgeon and support the rest of the components of system 1300. Due to the positioning of vertical support 1320, horizontal supports 1330, and surgeon support mechanism, a surgeon typically steps onto base 1310 from the back.

Mounted to base 1310 are vertical supports 1320. As illustrated, each of vertical supports 1320 includes a sleeve member 1322 and a vertical member 1324, which is received in sleeve member 1322. Vertical members 1324 may be moveable relative to sleeve members 1322 manually, electromechanically, or hydraulically. In manual embodiments, spring shocks or gas struts could be used to assist in movement, and sleeve members 1322 and vertical members 1324 may include suitable apertures for allowing the vertical members to be locked relative to the sleeve members.

At the top of each vertical support 1324 is a sleeve member 1326 for receiving one of horizontal supports 1330. Horizontal supports 1330 may be moveable relative to sleeve members 1326 manually, electromechanically, or hydraulically. In a manual embodiment, sleeve members 1326 and horizontal member 1330 may include suitable apertures for allowing the horizontal members to be locked relative to the sleeve members.

Coupled between horizontal supports 1330 is surgeon support mechanism 1340. As illustrated, surgeon support mechanism 1340 is a circular rod, which may or may not rotate, but surgeon support mechanism 1340 could have other configurations in other embodiments (e.g., an axle with an attached surgeon support). A variety of pads may be coupled to surgeon support mechanism 1340. The pads could, for example, be similar to those previously discussed herein or ones (e.g., cylindrical, square, oval, rectangular, etc.) that could be slid on before surgeon support mechanism 1340 is attached or ones that are separated (e.g., by having one or more slits therethrough) to allow installation after surgeon support mechanism 1340 is in place. Separated pads could, for example, be held together by straps, which could be secured by hook and loop fasteners.

Surgeon support mechanism 1340 is typically at least 24 inches wide to accommodate surgeons of various sizes. However, surgeon support mechanism 1340 may be much wider than this (e.g., 36 inches or 48 inches). Having a wider surgeon support mechanism allows the surgeon to move along the patient and to assume various angles (e.g., for sight and surgical technique) during an operation.

In operation, surgeon support mechanism 1340 can be adjusted in both height, by adjusting vertical members 1324 in sleeves 1322, and proximity to the patient, by adjust horizontal members 1330 in sleeves 1326. These adjustment may be performed in series or in parallel.

Surgeon support system 1300 has a variety of features. For example, system 1300 keeps the sterile field as free as possible of its components. This allows the surgeon to have improved sight lines and mobility of his/her arms. Additionally, surgeon support system 1300 provides improved support for surgeon support mechanisms that are longer in length (e.g., 36 inches).

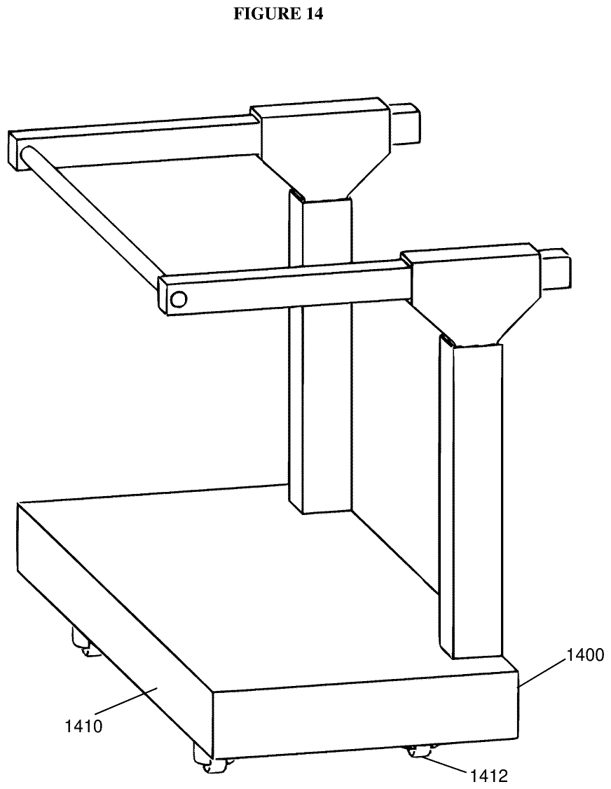

FIG. 14 illustrates an additional example surgeon support system 1400. Surgeon support system 1400 is similar to surgeon support system 1300 except that base 1410 includes wheels 1412. Wheels 1412 allow surgeon support system to me moved around the operating room. When note in use, wheels 1412 may be locked or retracted inside of base 1410.

FIG. 15 illustrates an a further example surgeon support system 1500. Surgeon support system 1500 is similar to surgeon support system 1300 except that base 1510 includes forks 1512 to provide stability to system 1500.

In particular procedures, a surgeon may have to lean far out on the surgeon support mechanism, which could cause a tipping problem. Forks 1512 help to ameliorate that. Forks 1512 may be permanently positioned relative to base 1500 or retractable thereinto.

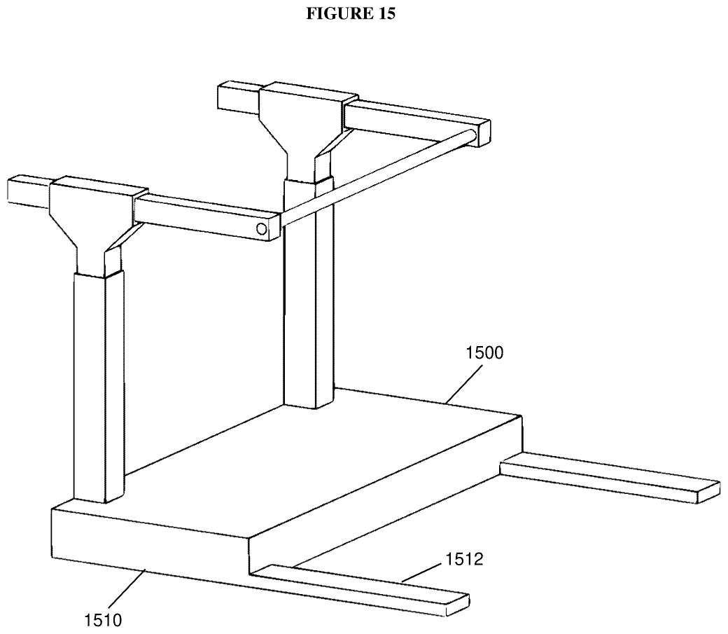

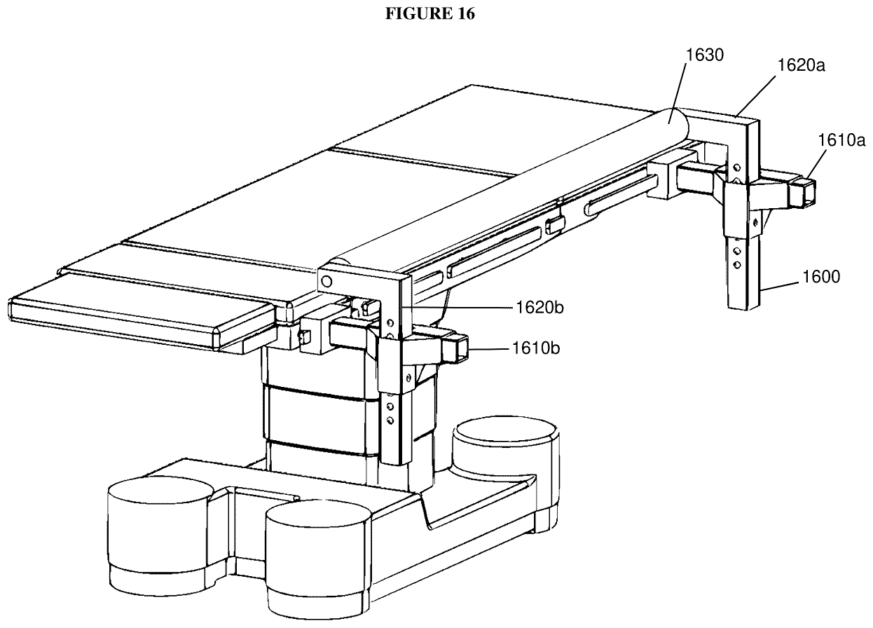

FIG. 16 shows an additional example embodiment of a surgeon support system 1600. Surgeon support system 1600 includes two base systems 1610, two vertical supports 1620, and a surgeon support mechanism 1630. Base systems 1610, vertical supports 1620, and surgeon supports mechanism 1630 may be configured as and use any of the adjustments discussed previously, like those for surgeon support system 100.

Unlike surgeon support mechanism 130 in surgeon support system 100, however, surgeon support mechanism 1630 is supported on both ends by vertical supports 1620. This provides increased stability for the surgeon support mechanism, which is especially useful with the mechanism is wide (e.g., 36 inches). Additionally, a double-sided support may improve the weight carrying capacity of the surgeon support system.

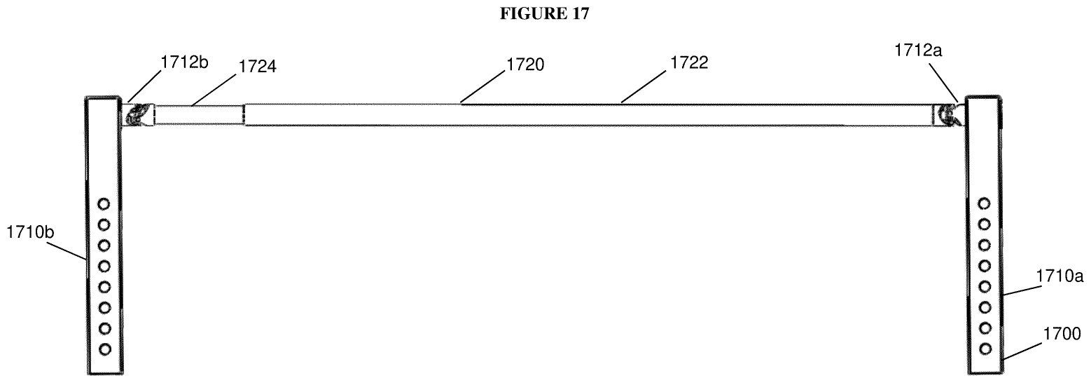

FIG. 17 illustrates selected components of an additional example surgeon support system 1700. Among other things, surgeon support system 1700 includes two vertical supports 1710 and a surgeon support mechanism 1720. Surgeon support mechanism 1720 is supported on both ends by vertical supports 1710.

At the top of each vertical support 1710 is a universal joint 1712. Surgeon support mechanism 1720 is coupled to universal joints 1712 at each end. Additionally, surgeon support mechanism 1720 includes a first member 1722 and a second member 1724. First member is configured to slidably receive second member 1724 to allow the length of surgeon support mechanism 1720 to be adjusted.

The illustrated components of surgeon support system 1700 may be useful for surgeon support systems where the surgeon support mechanism is supported on both ends, like in surgeon support system 1300 or surgeon support system 1600. If vertical supports like vertical supports 1710 are raised/lowered at different times, which might often be the case for manual adjustments, this would cause the entrained surgeon support mechanism 1720 to have to rotate relative the vertical members and elongate, which might be difficult to achieve if the surgeon support mechanism was a solid piece. With surgeon support system 1700, however, surgeon support mechanism 1720 may rotate relative to each vertical support 1710 and elongate if one of the vertical supports 1710 is raised/lowered at a time. In certain uses, having one vertical support higher than the other may be preferred (e.g., two people of different height are using the system). Additionally, surgeon support system 1700 may be useful if one end of a vertical support is closer to the operating table than another, creating a horizontally angle to the side of the operating table, which a surgeon might prefer if his/her torso is not parallel with the side rails of the operating table. Moreover, there are a variety of combinations of angles (e.g., horizontal and vertical) for which surgeon support system may be useful.

The pads, if any, for support mechanism 1720 pads could, for example, be similar to those previously discussed herein or ones (e.g., cylindrical, square, oval, rectangular, etc.) that which could be slid on before surgeon support mechanism 1720 is attached or ones that are separated (e.g., by having one or more slits therethrough) to allow installation after surgeon support mechanism 7120 is in place. Separated pads could, for example, be held together by straps, possible attached with hook and loop fasteners. In some implementations, the pad could expand to fill more space as the support mechanism 1720 telescopes outwardly.

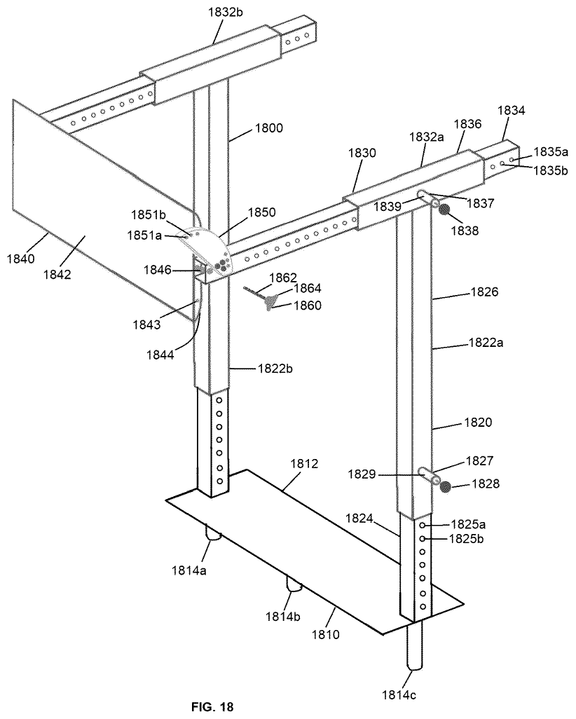

FIG. 18 illustrates another example surgeon support system 1800. System 1800 includes a base system 1810, a vertical support system 1820, a horizontal support system 1830, and surgeon support mechanism 1840.

Base system 1810 includes a base plate 1812 and rods 1814. Base plate 1812 provides vertical support to system 1800 and may be on top of or slightly recessed in a floor. Base plate 1812 should generally allow surgical items to be wheeled thereover and not be a trip hazard. The surgeon may stand on base plate 1812 during operation or between base plate 1812 and surgeon support system 1840. (The exact position of the surgeon will vary with the procedure being performed and the placement of horizontal support system 1830.) Thus, base plate 1812 is typically at least as wide as a human body at the hips (e.g., 18-24 inches), but may be wider in certain embodiments (e.g., 36 inches or 48 inches) to allow for surgeon movement. Rods 1814 are inserted into holes in a floor to provide horizontal and rotational stability to system 1800. In the illustrated implementation, rods 1814 are about 4 inches in length, but may be other lengths in other implementations. In certain implementations, base plate 1812 may have apertures therein for receiving screws to further secure base plate 1812 to the floor.

Vertical support system 1820 is attached to base plate 1812. In the illustrated implementation, vertical support system 1820 include two vertical supports 1822, but may include other numbers of vertical supports in other implementations.

Each vertical support 1822 includes an inner member 1824 and an outer member 1826, which are able to slide relative to each other. In the illustrated implementation, inner member 1824 is coupled (e.g., welded) to base plate 1812, and outer member 1826 slides relative to inner member 1824 when base system 1810 is mounted in a floor. In other implementations, outer member 1826 could be coupled to base plate 1812, and inner member 1824 could slide relative to outer member 1826. Inner member 1824 and outer member 1826 are shown to be square in cross section, but could have other cross sections (e.g., rectangular, elliptical, or circular).

To secure outer member 1826 relative to inner member 1824, inner member 1824 includes a series of apertures 1825, and outer member includes a pin mechanism 1827. Pin mechanism has a user-actuable knob 1828 and a pin (not viewable), which slides into and out of a sleeve 1829. By pulling on knob 1828, a user may retract the pin, which may be biased outward (e.g., by a spring) and slide outer member 1826 relative to inner member 1824. When outer member 1826 is at the appropriate position relative to outer member 1824, the user may push (or release) knob 1828, allowing the pin to extend from sleeve 1829 into one of apertures 1825.

Horizontal support system 1830 is coupled to vertical support system 1820. In the illustrated implementation, horizontal support system 1820 include two horizontal supports 1832, but may include other numbers of horizontal supports in other implementations.

Each horizontal support 1832 includes an inner member 1834 and an outer member 1836, which are able to slide relative to each other. In the illustrated implementation, outer member 1836 is coupled (e.g., welded) to a vertical support mechanism 1822, and inner member 1834 slides relative to outer member 1836. In other implementations, inner member 1834 could be coupled a vertical support 1822, and outer member 1836 could slide relative to inner member 1834. Inner member 1834 and outer member 1836 are shown to be square in cross section, but could have other cross sectional shapes in other implementations (e.g., rectangular, elliptical, or circular).

To secure inner member 1834 relative to outer member 1836, inner member 1834 includes a series of apertures 1835, and outer member includes a pin mechanism 1837. Pin mechanism 1837 has a user-actuable knob 1838 and a pin (not viewable), which slides into and out of a sleeve 1839. In certain implementations, the pin may be biased outward (e.g., by a spring.) By pulling on knob 1838, a user may retract the pin into sleeve 1837 and slide inner member 1834 relative to outer member 1836. When inner member 1834 is at the appropriate position relative to outer member 1836, the user may push (or release) knob 1838, allowing the pin to extend from sleeve 1839 into one of apertures 1835.

Surgeon support mechanism 1840 is coupled to horizontal supports 1832 near one of their ends. As illustrated, surgeon support system 1840 includes a frame 1842 and a pad 1844. In particular implementations, pad 1844 may be removable from frame 1842.

In the illustrated implementation, frame 1842 is rotatable about its longitudinal axis. To accomplish this, axles 1846 (only one of which is visible) extend from frame 1842 and rotatably mount in horizontal support mechanisms 1832. Axles 1846 may, for example, be welded to frame 1842. Mounting a pin in a horizontal support mechanism may, for example, be accomplished with bushings (e.g., one on each side of the horizontal support mechanism). In certain implementations, the pins may be secured in the bushings by having a screw inserted therethrough.

To secure frame 1842 from rotating, frame 1842 includes a number of apertures 1843 (only one which is viewable in FIG. 18) therein. Additionally, system 1800 includes a locking mechanism 1850 that is coupled (e.g., by screws or rivets) to one of horizontal supports 1832. Locking mechanism 1850 also includes a series of apertures 1851 therein that are aligned with the radial sweep of apertures 1843. In other implementations, system 1800 could include multiple locking mechanisms.

In the illustrated implementation, the hidden one of apertures 1843 is aligned during rotation with the upper ones of apertures 1851, and the viewable one of apertures 1843 is aligned during rotation with the lower ones of apertures 1851. Various combinations of apertures 1843 and apertures 1851 may be used in other implementations.

A locking device 1860 may be inserted through one of apertures 1851 and into one of apertures 1843 to secure frame 1842 from rotating. Locking device 1860 includes a stud 1862 and a knob 1864. Stud 1862 may or may not be threaded. If the stud is threaded, apertures 1851 and/or aperture 1843 may also be threaded to receive the threads.

Surgeon support mechanism 1840 may be used in a variety of orientations. For example, as illustrated, frame 1842 may be in a vertical orientation, in which case the surgeon could lean on pad 1844 with their torso. In other orientations, frame 1842 may be tilted slightly off of vertical to provide support for different procedures. However, in other orientations, frame 1842 may be horizontal (e.g., to support a surgeons wrists or forearms). Frame 1842 could also be tilted slightly above over below horizontal. A variety of orientations are possible in various implementations. In some uses, the surgeon may even want frame 1842 to rotate freely. This could, for example, be useful in the vertical leaning position.

The ability to vertically, horizontally, and rotatably adjust surgeon support mechanism 1840 provides a variety of uses for system 1800. For example, the surgeon support mechanism may support the surgeon in the primary positions in which a surgeon may perform surgery. The first is a standing position in which the surgeon's stomach, chest, and/or pelvic area can lean on the rotatable support system, typically when locked in a vertical position. The second is also a standing position in which the surgeon's wrists or forearms are resting on the rotatable support system, typically when locked in a horizontal position. The third is a seated position in which the surgeon's wrists or forearms are resting on the rotatable support system, typically in a horizontal position. There are also a variety of other positions in which a surgeon can be and use system 1800.

Surgeon support system 1800 may be composed primarily of metal (e.g., aluminum or stainless steel), hard plastic or composite, although some portions (e.g., knobs and pads) may be composed of flexible plastic (e.g., vinyl). Surgeon support system 1800 may generally be composed of any material that resists degradation and is easy to clean.

In other implementations, vertical supports 1822 and horizontal supports 1832 may be moveable relative to by automated means (e.g., electromechanically or hydraulically). Moreover, in manual embodiments, spring shocks or gas struts could be used to assist in movement. Additionally, other locking mechanisms could be used to secure surgeon support mechanism 1840 (e.g., a pin in axle or a Hirth joint). Moreover, base plate 1812 may not be used (e.g., if vertical supports 1820 directly engage the floor).

Although system 1800 has primarily been discussed in relation to supporting a surgeon, system 1800 may have a variety of other uses. For example, system 1800 may be useful for factory workers, machinists, or any other occupation that requires extended manual manipulation with the hands while standing or seated.

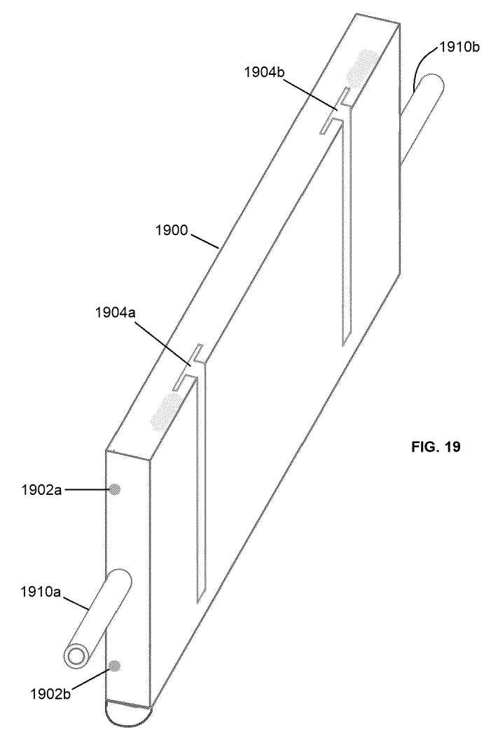

FIG. 19 illustrates an example frame 1900 for a surgeon support system. Similar to frame 1842, frame 1900 includes apertures 1902 to allow frame 1900 to be secured in various rotational positions. Extending from frame 1900 are axles 1910, around which frame 1900 may rotate.

Frame 1900 also includes slots 1904. Slots 1904 allow pads, like pad 1844, to be coupled and decoupled from frame 1900. For example, a pad may be decoupled from frame 1900, cleaned, and recoupled. As another example, pads of various orientations may be coupled to frame 1900 depending on the needs of the surgeon.

FIG. 20 illustrates an example pad 2000 that may be used for a rotatable support system. As illustrated, pad 2000 includes rails 2002 that may be inserted into slots of a frame of a surgeon support mechanism. In certain implementations, rails 2002 may be locked into place relative to slots 1904. Pins, latches, ball spring plungers, or any other releasable detent could be used.

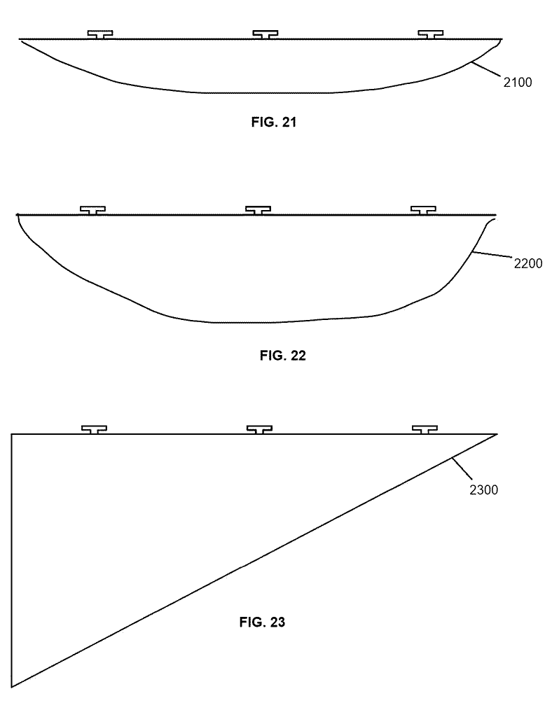

FIGS. 21-23 illustrate additional example pads that may be used for a surgeon rotatable support mechanism. Pad 2100 is relatively thin, which might be a surgeon's preference for the horizontal orientation Pad 2200 is thicker, which might be a surgeon's preference for the vertical orientation. And pad 2300 is angled, which could be useful in the vertical orientation if a surgeon does not want to be exactly parallel to the operating table. Other configurations may be used in other implementations.

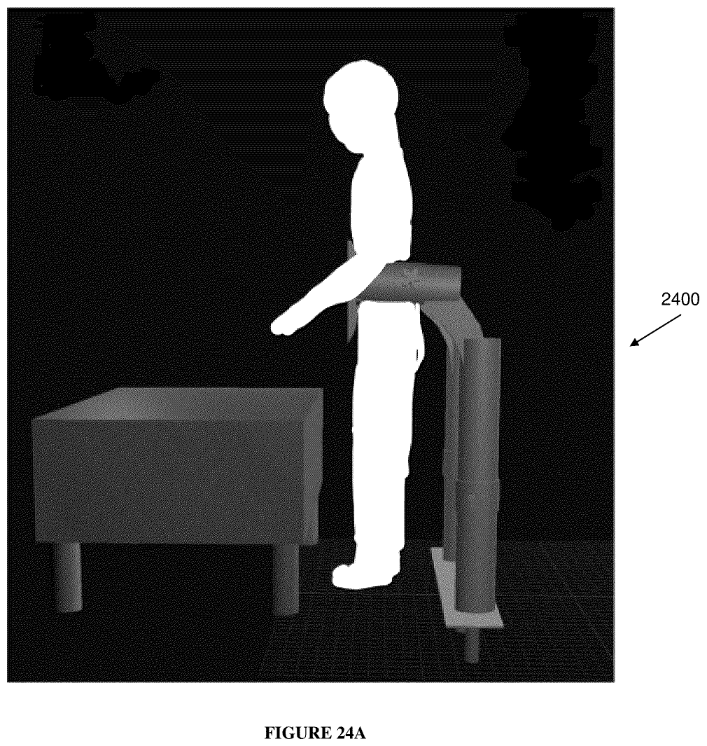

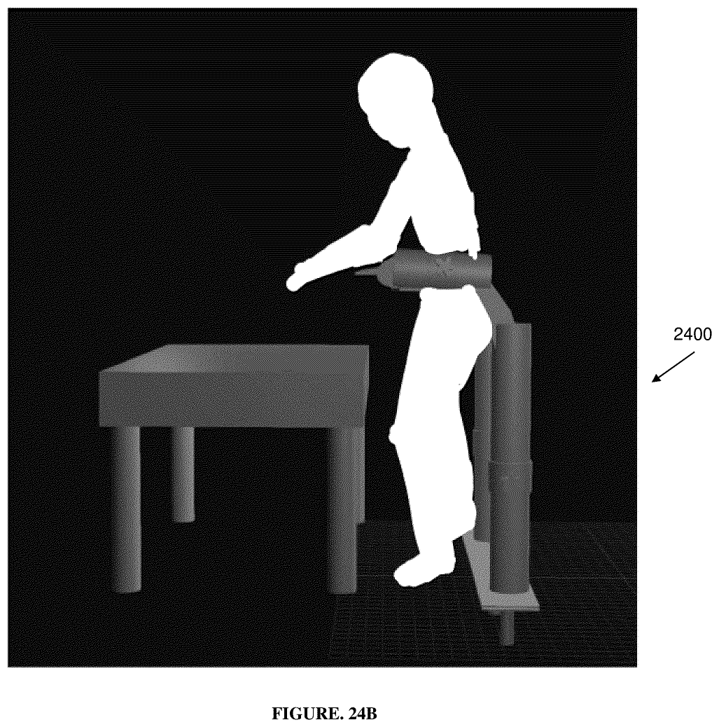

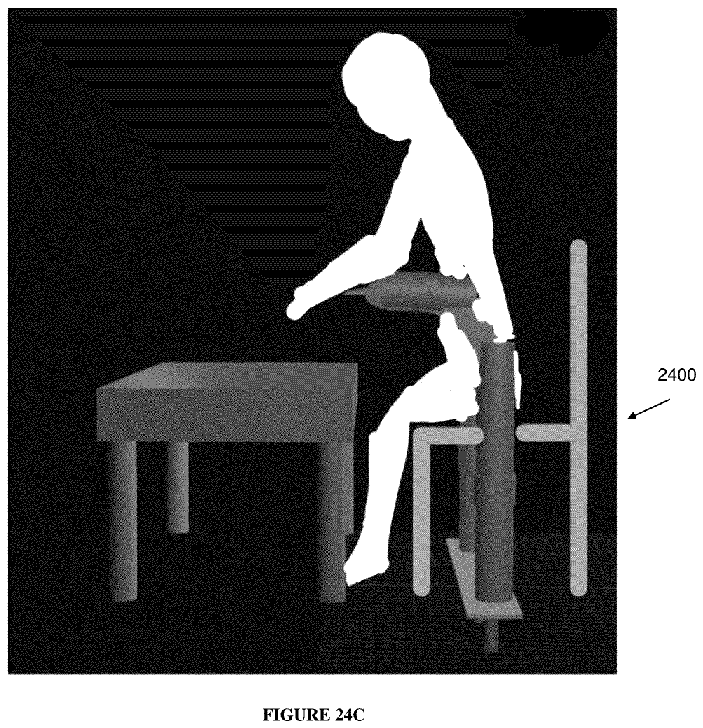

FIGS. 24A-24C illustrate another example surgeon support system 2400 in use. In FIG. 24A, the surgeon is using the system in a standing position, with the surgeon support mechanism in a vertical orientation. In FIG. 24B, the surgeon is using the system in a crouched position, with the surgeon support mechanism in a horizontal position. In FIG. 24C, the surgeon is using the system in a seated position, with the surgeon support mechanism pad in a horizontal position. The other surgeon support systems can be used in a similar manner.

System 2400 could also have a variety of other uses. For example, system 2400 may be useful for factory workers, machinists, or any other occupation that requires extended manual manipulation with the hands while standing or seated.

The drawings shown herein are believed to be fairly accurate. Thus, approximate lengths, angles, and relative sizes between parts can be extracted therefrom by one of ordinary skill in the art. However, the invention is not limited to the exact dimensions/sizes shown. Moreover, if one object is “near” another object or part of the other object, the first object may be within 33% of the longest dimension of the second object.

A number of implementations have been illustrated and discussed, and several others have been mentioned or suggested. Moreover, those of ordinary skill in the art will readily recognize that a variety of additions, deletions, substitutions, and transformations may be made while still achieving a surgeon support system. Thus, the scope of protected matters should be judged based on the claims, which may encompass one or more aspects of one or more implementations.

1. A surgeon support system comprising:
a base system attachable to a side rail of an operating table, the base system comprising:
a clamp member adapted to be slidably received on an operating table side rail,
an elongated member coupled to the clamp member at a first end and extending laterally away therefrom in a direction perpendicular to a received side rail to a second end, and
a sleeve mechanism coupled to the elongated member distally from the clamp member, the sleeve mechanism including a vertical sleeve;
a vertical support comprising a vertical member having a first end configured to be received by the vertical sleeve and extending upward from the first end to a second end distal from the sleeve; and
a surgeon support mechanism coupled to the vertical support and extending laterally therefrom in a direction perpendicular to the vertical member.

2. The surgeon support system of claim 1, wherein the vertical support comprises a horizontal member having a first end near the second end of the vertical member, the horizontal member extending laterally toward the clamp member to a second end, wherein the surgeon support is coupled to the horizontal member near its second end.

3. The surgeon support system of claim 2, wherein the horizontal member is hinged at a midpoint to allow the second end of the horizontal member to rotate laterally relative to the first end of the horizontal member.

4. The surgeon support system of claim 1, wherein the surgeon support mechanism comprises an axle and a surgeon support coupled to the axle.

5. The surgeon support system of claim 4, wherein the surgeon support is attached to the axle, and the vertical support is configured to receive an end of the axle such that the surgeon support can rotate relative to vertical support, the vertical support comprising a rotational locking mechanism to fix the axle at different angles of rotation.

6. The surgeon support system of claim 5, wherein the vertical support comprises a horizontal member having a first end near the second end of the vertical member, the horizontal member extending laterally toward the clamp member to a second end, wherein the rotational locking mechanism is located near the second end of the horizontal member.

7. The surgeon support system of claim 1, wherein the sleeve mechanism comprises a sleeve configured to receive the elongated member and slide relative thereto, the sliding of the sleeve mechanism relative to the elongated member moving the vertical support relative to the clamp member.

8. The surgeon support system of claim 1, wherein the base system is configured such that the elongated member can rotate relative to the clamp member about an axis between the elongated member's ends.

9. The surgeon support system of claim 8, wherein the first end of the elongated member comprises a disc perpendicular to the axis, and the clamp member include a circular shell for receiving the disc.

10. The surgeon support system of claim 1, wherein the clamp member comprises a lateral slot therethrough, and the system further comprises a latch member, the latch member having a first end adapted to receive an end of an operating table side rail and a thin, elongated body that extends through the slot in the clamp member, the elongated body having a plurality of apertures for receiving pins therein to prevent the clamp member from sliding relative to the latch member.

11. The surgeon support system of claim 1, wherein base system is configured such that the elongated member can rotate vertically relative to the clamp member about the elongated member's first end.

12. The surgeon support system of claim 11, wherein the clamp member includes a pivot point, and the first end of the elongated member is configured to be coupled to the pivot point.

13. The surgeon support system of claim 1, further comprising:
a second base system attachable to a side rail of an operating table, the base system comprising:
a second clamp member adapted to be slidably received on a side rail of an operating table,
a second elongated member coupled to the second clamp member at a first end and extending laterally away therefrom in a direction perpendicular to a received side rail to a second end, and
a second sleeve mechanism comprising a vertical sleeve coupled to the elongated member distally from the second clamp member;
a second vertical support comprising a second vertical member having a first end configured to be received by the second vertical sleeve and extending vertically away from the second sleeve to a second end distal from the second sleeve; and
wherein the surgeon support is also attached to the second vertical support and extends laterally therefrom in a direction perpendicular to the second vertical support and towards the first vertical support.

14. The surgeon support system of claim 13, wherein the first vertical support and the second vertical support are rotatably coupled to an axle of the surgeon support mechanism.

15. The surgeon support system of claim 13, wherein the axle comprises a telescoping rod, the first vertical support comprises a universal joint coupled to one end of the telescoping rod, and the second vertical support comprises a universal joint coupled to another end of the telescoping rod.

16. A surgeon support system comprising:
a base upon which a surgeon can stand and configured to allow a surgeon to move laterally;
a vertical support system coupled to the base, the vertical support system having a vertical member that may raise and lower relative to the base;
a horizontal member coupled to the vertical member near a first end and extending laterally across the base to a second end; and
a surgeon support mechanism coupled to the horizontal member near the second end and extending laterally therefrom in a direction perpendicular to the vertical member.

17. The surgeon support system of claim 16, wherein the surgeon support mechanism includes an axle and a surgeon support coupled to the axle

18. The surgeon support system of claim 16, wherein the vertical support system includes a sleeve for receiving the horizontal member, the horizontal member being horizontally adjustable by sliding through the sleeve.

19. The surgeon support system of claim 16, wherein the surgeon support mechanism is rotatably coupled to the horizontal member.

20. The surgeon support system of claim 19, wherein horizontal member includes a pivot point near the second end, and one end of the surgeon support mechanism engages with the pivot point.

21. The surgeon support system of claim 16, wherein the base includes wheels for moving the base relative to a floor.

22. The surgeon support system of claim 16, further comprising:
a second vertical support system coupled to the base, the second vertical support system having a second vertical member that may raise and lower relative to the base; and
a second horizontal member coupled to the second vertical member;
wherein the surgeon support is coupled to the second horizontal member at a second end.

23. The surgeon support system of claim 22, wherein the first horizontal member and the second horizontal member are rotatably coupled to the surgeon support.

24. The surgeon support system of claim 23, wherein the axle comprises a telescoping rod, the first horizontal member comprises a universal joint coupled to one end of the telescoping rod, and the second horizontal member comprises a universal joint coupled to another end of the telescoping rod.

25. A surgeon support system comprising:
a first vertical support system, the first vertical support system having a first vertical member that may raise and lower;
a first horizontal member coupled to the first vertical member;
a second vertical support system, the second vertical support system having a second vertical member that may raise and lower;
a second horizontal member coupled to the second vertical member; and
a surgeon support mechanism coupled to the first horizontal member at one end and to the second horizontal member at a second end, the surgeon support extending laterally in a direction perpendicular to the vertical members and including an axle and a surgeon support coupled to the axle.

26. The surgeon support system of claim 25, wherein each vertical support system includes a sleeve for receiving a respective horizontal member, the horizontal members being horizontally adjustable by sliding through the sleeves.

27. The surgeon support system of claim 25, wherein the surgeon support is rotatable relative to the horizontal members.

28. The surgeon support system of claim 27, wherein the axle comprises a telescoping rod, the first horizontal member comprises a universal joint coupled to one end of the telescoping rod, and the second horizontal member comprises a universal joint coupled to another end of the telescoping rod.
